(19) United States
(12) Patent Application Publication
(10) Pub. No.: US 2024/0293133 A1
(43) Pub. Date: Sep. 5, 2024

(54) MEDICAL GRASPING DEVICE WITH SLIDE ACTUATOR

(71) Applicant: Merit Medical Systems, Inc., South Jordan, UT (US)

(72) Inventors: Richard D. Jenkins, Kennett Square, PA (US); Michael Dean Haslam, Sandy, UT (US); Nicholas Accisano, III, Howell, NJ (US); Nicole Willardson, Herriman, UT (US); Gregory R. McArthur, Sandy, UT (US)

(21) Appl. No.: 18/591,252

(22) Filed: Feb. 29, 2024

Related U.S. Application Data
(60) Provisional application No. 63/488,098, filed on Mar. 2, 2023.

(51) Int. Cl. A61B 17/22; A61B 17/00

(52) U.S. Cl. CPC A61B 17/22031; A61B 17/00234; A61B 2017/003; A61B 2017/00367; A61B 2017/00862

(57) ABSTRACT

Devices, systems, and methods used to grasp and retrieve a target foreign body or object from a body lumen are disclosed. The devices include a slide actuator, a catheter operably coupled to the slide actuator, and a grasping member in a fixed position relative to the slide actuator. The slide actuator can axially displace the catheter relative to the grasping member to transition the grasping member from an open state to a closed or grasping state. The slide actuator can be locked in the closed state to retrieve the target foreign object from the patient's body.

### RELATED APPLICATIONS

This application claims priority to U.S. Provisional Application No. 63/488,098, filed on Mar. 2, 2023 and titled, “Medical Grasping Device with Slide Actuator,” which is hereby incorporated by reference in its entirety.

### TECHNICAL FIELD

The present disclosure relates generally to medical grasping or retrieving devices. More specifically, the present disclosure relates to medical grasping or retrieving devices configured for retrieval of objects from body lumens and methods of use.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a grasping or retrieval device.

FIG. 2 is a perspective exploded view of an embodiment of a slide actuator of the grasping device of FIG. 1.

FIG. 3 is a perspective view of an embodiment of a slide button of the grasping device of FIG. 1.

FIG. 4 is a perspective view of an embodiment of a locking member of the grasping device of FIG. 1.

FIG. 5 is a perspective view of an embodiment of a slide carriage of the grasping device of FIG. 1.

FIG. 6 is a perspective view of an embodiment of an assembly of a portion of the components of the grasping device of FIG. 1.

FIG. 7A is a perspective view of an embodiment of a grasping or retrieving member in an open state of the grasping device of FIG. 1.

FIG. 7B is a perspective view of the grasping member of FIG. 7A in a closed or grasping state.

FIG. 8A is a perspective cross-sectional view of an embodiment of a distal portion of the grasping device of FIG. 1 with the grasping member in the open state.

FIG. 8B is a perspective cross-sectional view of the distal portion of the grasping device of FIG. 8 in the closed state.

FIG. 9 is a perspective view of the grasping device of FIG. 1 in use.

FIG. 10 is a perspective view of an embodiment of a grasping or retrieving device.

### DETAILED DESCRIPTION

The various embodiments disclosed herein generally relate to medical grasping or retrieving devices. More specifically, the various embodiments relate to medical grasping or retrieving device systems, for example, medical grasping or retrieving device assemblies and related methods. In some embodiments, the medical grasping or retrieving device assembly comprises a catheter, a grasping or retrieving member having a plurality of resilient prongs coupled to an elongate member, and a slide actuator. The slide actuator can allow the grasping member to transition from an open state to a closed or grasping state while maintaining longitudinal stagnation by longitudinal displacement of the catheter over the grasping member. Also disclosed herein are methods of utilizing a medical grasping or retrieving device assembly.

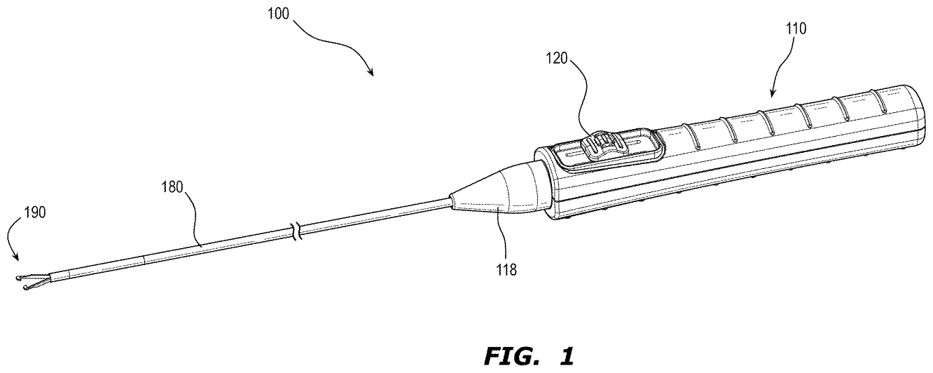

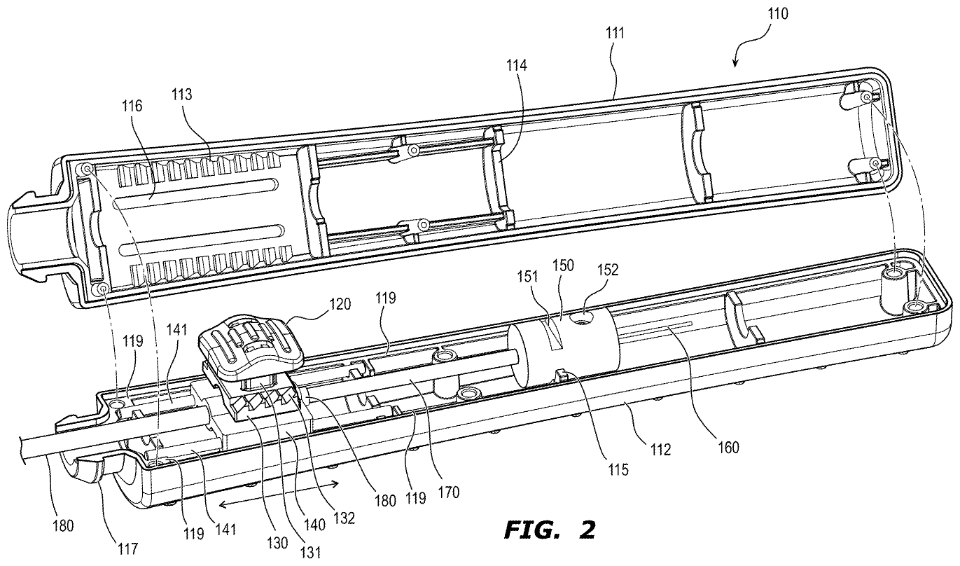

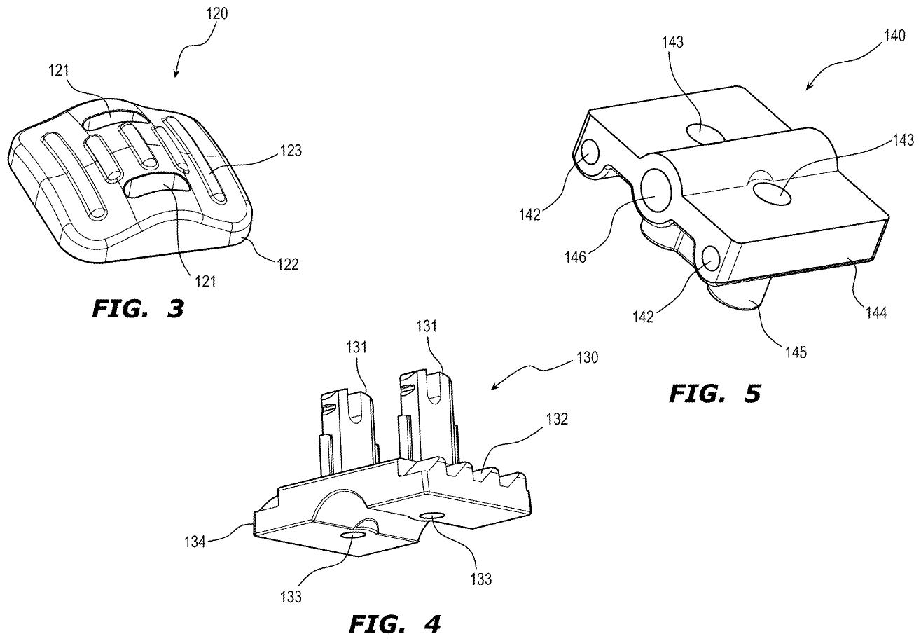

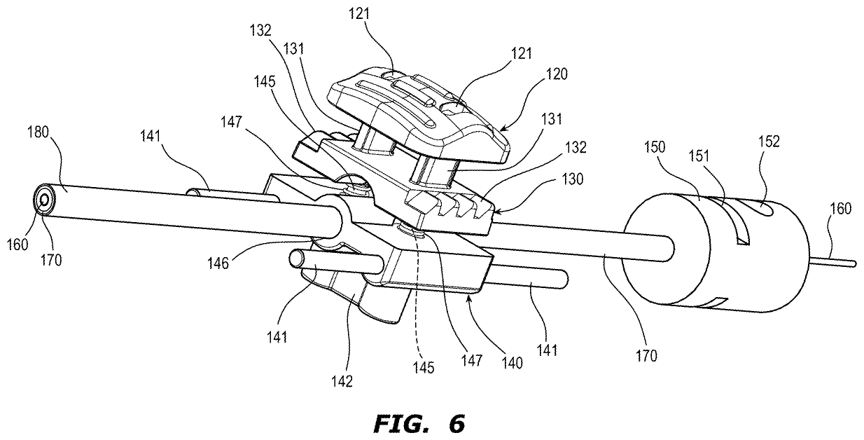

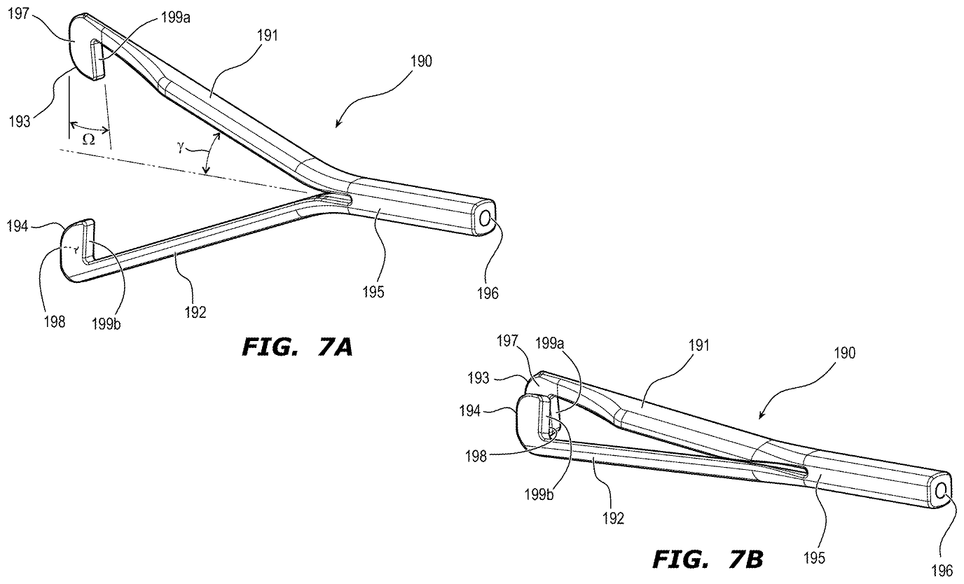

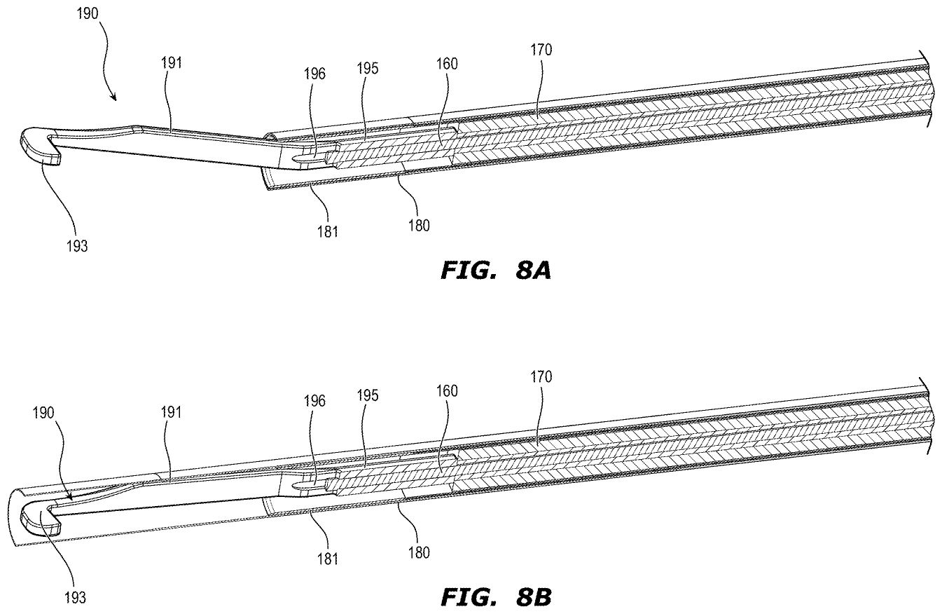

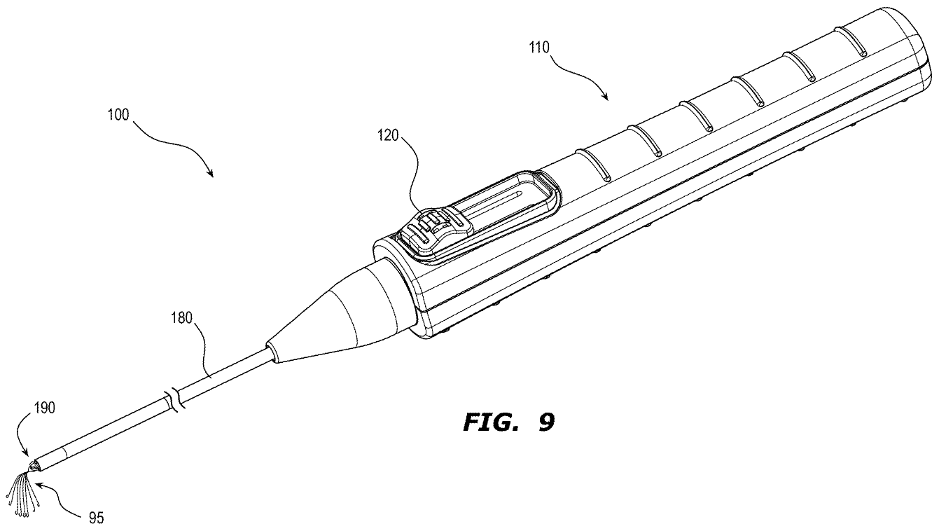

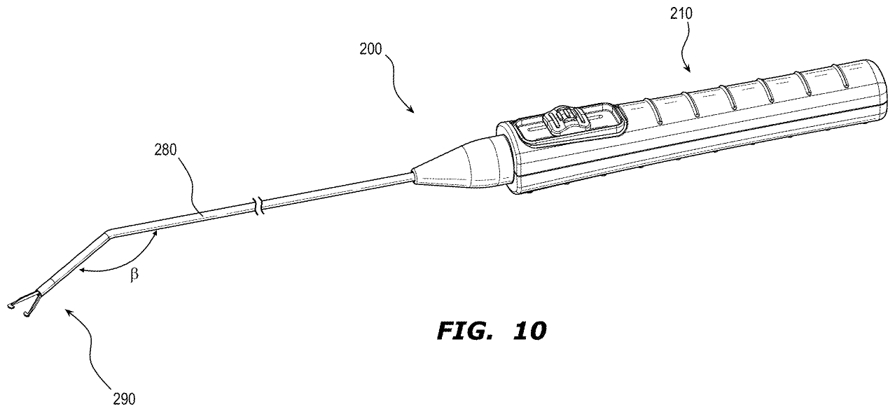

FIG. 1 illustrates an embodiment of a grasping or retrieval device. FIG. 2 illustrates an embodiment of a slide actuator of the grasping device. FIG. 3 illustrates an embodiment of a slide button of the grasping device. FIG. 4 illustrates an embodiment of a locking member of the grasping device. FIG. 5 illustrates an embodiment of a slide carriage of the grasping device. FIG. 6 illustrates an embodiment of an assembly of a portion of the components of the grasping device. FIG. 7A illustrates an embodiment of a grasping or retrieving member in an open state of the grasping device. FIG. 7B illustrates the grasping member in a closed or grasping state. FIG. 8A illustrates an embodiment of a distal portion of the grasping device with the grasping member in the open state. FIG. 8B illustrates the distal portion of the grasping device in the closed state. FIG. 9 illustrates the grasping device in use. FIG. 10 illustrates another embodiment of a grasping or retrieving device. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIG. 1 illustrates an embodiment of a medical grasping or retrieving device 100. As depicted in FIG. 1, the medical grasping device 100, can include a slide actuator 110, a catheter 180 extending distally from the slide actuator 110, and a grasping or retrieving member 190 extending distally from the catheter 180. In some embodiments, as shown in FIG. 1, the medical grasping device 100 may include a strain relief 118 coupled to a distal end of the slide actuator 110 and disposed concentrically over the catheter 180. The strain relief 118 may prevent kinking of the catheter 180 at the interface of the slide actuator 110. Kinking of the catheter 180 can prevent proper function of the medical grasping device 100.

FIG. 2 illustrates an exploded view of the slide actuator 110. As depicted in FIG. 2, the slide actuator 110 can include a top housing 111 and a bottom housing 112 that can be coupled together using any suitable technique, such as snap fit, fasteners, gluing, bonding, welding, etc. In one embodiment, the top housing 111 and the bottom housing 112 may be in a clam shell configuration that includes a hinge and a snap fit closure. As shown, the top housing 111 includes locking teeth 113 configured to engage with locking teeth 132 of a locking member 130. The locking teeth 113, 132 may be configured to allow ratcheting of the locking member 130 when the locking member 130 is displaced distally and to prevent proximal movement of the locking member 130 when the locking teeth 113, 132 are engaged. In other embodiments, the locking teeth 113, 132 may be configured to engage such that distal and proximal displacement of the locking member 130 is prevented when the locking teeth 113, 132 are engaged. In the illustrated embodiment, the top housing 111 further includes at least one slide slot 116 disposed through the top housing 111. As shown in the embodiment of FIG. 2, the top housing may include two parallel slide slots 116. The slide slots 116 may be configured to receive slide posts 131 of the locking member 130 to permit proximal and distal movement of a slide button 120. The top housing 111 further includes an anchor brace 114 to engage with a brace groove or slot 151 of an anchor 150 coupled to an elongate member 160. The brace groove 151 can prevent longitudinal movement of the elongate member 160 when the medical grasping device 100 is displaced proximally to retrieve a foreign object from a patient's body. The top housing 111, may include grip enhancing features to allow enhanced gripping of the top housing 111 by a healthcare worker. For example, as shown in FIG. 1, the top housing 111 includes transverse ridges spaced along the length of the top housing 111. The top housing 111 may further include a plurality of cavities and walls as shown in FIG. 2 to facilitate stability and assembly of the medical grasping device 100. A strain relief fitting 117 may be disposed at a distal end of the top housing 111 and configured to facilitate coupling of the strain relief 118 to the top housing 111.

As depicted in the embodiment of FIG. 2, the bottom housing 112 can include an anchor brace 115, similar to the anchor brace 114 of the top housing 111 and configured to engage with the brace groove 151 of the anchor 150 as previously described. The bottom housing 112 can further include slide rail retainers 119 configured to receive and retain slide rails 141 from vertical and longitudinal movement. The bottom housing 112, may include grip enhancing features to allow enhanced gripping of the bottom housing 112 by a healthcare worker. For example, as shown in FIG. 2, the bottom housing 112 includes transverse ridges spaced along the length of the bottom housing 112. The bottom housing 112 may further include a plurality of cavities and walls as shown in FIG. 2 to facilitate stability and assembly of the medical grasping device 100. A strain relief fitting 117 may be disposed at a distal end of the bottom housing 112 and configured to facilitate coupling of the strain relief 118 to the bottom housing 112 and top housing 111.

FIG. 3 illustrates an embodiment of the slide button 120. As shown in FIG. 3, the slide button 120 includes a body 122 that is substantially rectangular in shape. At least one slide post passage 121 can be disposed through the body 122 from a top surface to a bottom surface. In the depicted embodiment of FIG. 3, two slide post passages 121 are disposed through the body 122. The slide post passage 121 can be configured to receive and engage with the slide posts 131 of the locking member 130 (See FIG. 4). This configuration can couple the slide button 120 and the locking member 130 such that when the slide button 120 is displaced longitudinally or vertically, the locking member 130 can be coincidingly displaced. In some embodiments, engaging members may be disposed within the slide post passage 121 to mate with engaging members of the slide posts 131 to lock the slide button 120 and the locking member 130 together. In certain embodiments, the slide button 120 can include grip enhancing features to facilitate longitudinal and vertical displacement of the slide button 120 when engaged by a finger of the healthcare worker. For example, as shown in FIG. 3, the slide button 120 includes a plurality of transversely oriented ribs 123 distributed on the top surface along a longitudinal axis of the body 122.

FIG. 4 illustrates an embodiment of the locking member 130. As depicted in FIG. 4, the locking member 130 can include a base portion 134, at least one slide post 131, and at least one set of locking teeth 132. In the embodiment of FIG. 4, the locking member 130 includes two slide posts 131 and two sets of locking teeth 132. The slide posts 131 extend vertically from the base portion 134 and are configured to extend through the slide slots 116 of the top housing 111 and to be received into the slide post passages 121 of the slide button 120 as previously described. The slide posts 131 can have a substantially rectangular cross-section that prevents rotation of the slide button 120. The depicted locking member 130 of FIG. 4 includes two sets of locking teeth 132. The locking teeth 132 can engage with the locking teeth 113 of the top housing 111. The locking teeth 113, 132 may be oriented such that when the locking teeth 113, 132 are engaged, the slide button 120 and the locking member 130 can be ratcheted distally and proximal displacement can be prevented. The locking member 130 can further include at least one post recess 133 configured to slidingly receive a post 145 (See FIG. 6). The illustrated embodiment of FIG. 4 includes two post recesses 133. The locking member 130 can be vertically displaced over the posts 145 to disengage the locking teeth 113, 132 when the catheter 180 is extended and retracted. In other words, the posts 145 can slide within the post recesses 133 as the locking member 130 is displaced vertically up and down.

FIG. 5 illustrates an embodiment of the slide carriage 140 of the medical grasping device 100. As illustrated in FIG. 5, the slide carriage 140 can include a base 144. At least one post passage 143 can be vertically disposed through the base 144 and extend through post receivers 148 that extend from a lower surface of the base 144. The depicted embodiment of FIG. 5 includes two post passages 143 and two post receivers 148. The post passages 143 can be configured to vertically receive the posts 145 such that the slide carriage 140 and the locking member 130 are coupled to move longitudinally together. The base 144 can further include at least one slide rail passage 142 configured to slidingly receive a slide rail 141 (See FIG. 6.) to allow longitudinal displacement of the slide carriage 140. The depicted embodiment of FIG. 5 includes two parallel oriented rail passages 142. The base 144 can further include a catheter receiver 146 longitudinally disposed through the base 144. The catheter 180 may be coupled to the catheter receiver 146 such that longitudinal movement of the slide carriage 140 can result in a corresponding longitudinal movement of the catheter 180.

FIG. 6 illustrates an assembly of the slide button 120, the locking member 130, and the slide carriage 140. The components can be configured to facilitate longitudinal displacement of the catheter 180 relative to an assembly of the elongate member 160, the filler tube 170, and the grasping member 190. As depicted in FIG. 6, the slide posts 131 of the locking member 130 are received into the slide post passages 121 of the slide button 120. In some embodiments, the slide posts 131 may lockingly couple with the slide button 120 to prevent decoupling of the slide button 120 from the locking member 130. The locking member 130 is slidingly disposed over posts 145 such that the locking member 130 and the slide button 120 are vertically displaceable relative to the slide carriage 140. Resilient members 147 (e.g., compression springs) may be disposed around the posts 145 and between the locking member 130 and the slide carriage 140. The resilient members 147 can provide an upwardly directed force to the locking member 130 such that the locking teeth 113, 132 are engaged when a downwardly directed force is released from the slide button 120 by the healthcare worker to lock the medical grasping device 100 in a longitudinal position. The slide rails 141 are shown slidingly disposed through the slide rail passages 142 to allow longitudinal movement of the slide carriage 140 over the slide rails 141. This configuration of the slide button 120, the locking member 130, and the slide carriage 140 may facilitate longitudinal movement of the slide button 120, the locking member 130, and the slide carriage 140 as an assembled unit. The configuration may also allow the slide button 120 and locking member 130 to move vertically relative to the slide carriage 140 and to be biased into a longitudinal locked state.

FIG. 6 depicts the filler tube 170 concentrically disposed at least partially within the catheter 180 and extending between the anchor 150 and the grasping member 190. The filler tube 170 can fill at least a portion of a lumen of the catheter 180 to prevent kinking of the catheter 180. The elongate member 160 is concentrically disposed within the filler tube 170 and extends proximally from the filler tube 170. The anchor 150 can be coupled to the elongate member 160 using any suitable technique. For example, in the embodiment shown in FIG. 6, the anchor 150 is coupled to the elongate member 160 using a fastener 152 (e.g., set screw). This configuration facilitates longitudinal fixation of the filler tube 170 and the elongate member 160 relative to the catheter 180.

FIG. 7A illustrates an embodiment of the grasping or retrieving member 190 of the medical grasping device 100 in an open state. As shown in FIG. 7A, the grasping member 190 includes a first resilient prong 191 and a second resilient prong 192, both extending from a stem 195. In other embodiments, the grasping member 190 may include three, four, five, or more resilient prongs. The grasping member 190 can be formed from any suitable resilient metal material. For example, the grasping member 190 can be formed from a shape memory nickel-titanium alloy (e.g., NITINOL). In other embodiments, the grasping member may be formed from any suitable grade of stainless steel. The stem 195 includes a bore 196 open at a proximal end. The bore 196 can be sized to receive a distal end of the elongate member 160 to facilitate coupling of the grasping member 190 and the elongate member 160. The coupling may be achieved using any suitable technique that allows a separation pull force of at least 10 lbf, such as peening, crimping, welding, bonding, etc.

The first resilient prong 191 and the second resilient prong 192 can each form an angle γ in the open state relative to a longitudinal axis of the grasping member 190. The angle γ can range from about 10 degrees to about 60 degrees, about 10 degrees to about 45 degrees, from about 30 degrees to about 60 degrees, from about 35 degrees to about 45 degrees, and may be about 40 degrees. The angle γ can define a distance between the distal ends of the resilient prongs 191, 192 to determine a size of the target foreign object to be grasped. For example, in some embodiments, when the angle γ is about 40 degrees the distance between the distal ends is about 0.19 inch and can facilitate grasping the target foreign object having a grasping dimension of up to about 0.19 inch. When γ is greater than 40 degrees the distance is greater than 0.19 inch and when γ is less than 40 degrees the distance is less than 019 inch. Additionally, the angle γ can be defined by the stress and strain limits applied to the resilient prongs 191, 192 when in the closed state to prevent breakage of the resilient prongs 191, 192. For example, when γ is greater than about 60 degrees the stress and strain applied to the resilient prongs 191,192 may exceed a fracture limit of the material of the resilient prongs 191,192.

The first resilient prong 191 includes a first grasping or retrieval feature 193 disposed at a distal end and extending inwardly toward a longitudinal axis of the grasping member 190. The first grasping feature 193 includes a first face or surface 197. The second resilient prong 192 includes a second grasping or retrieval feature 194 disposed at a distal end and extending inwardly toward the longitudinal axis of the grasping member 190. The second grasping feature 194 includes a second face or surface 198. When the grasping member 190 is in a closed state, as shown in FIG. 7B, the first and second grasping features, 193, 194 slide by one another such that the first and second faces 197, 198 are disposed adjacent each other with the faces oriented toward or facing one another. In certain embodiments, the first and second faces 197, 198 may be in contact with one another when the grasping member 190 is in the closed state. The first grasping feature 193 includes a first proximal face or surface 199a and the second grasping feature 194 includes a second proximal face or surface 199b. The first and second proximal faces 199a, 199b are oriented proximally at an angle Ω relative to a perpendicular axis to the longitudinal axis of the grasping member 190. In some embodiments, the angle Ω may range from about negative 5 degrees to about 20 degrees, from about zero degree to about 10 degrees, and may be about 5 degrees. The proximal orientation of the proximal faces 199a, 199b can facilitate secure grasping and retrieval of a target foreign body at a pull force of at least 10 lbf. and may facilitate further closure of the resilient prongs 191, 192 by applying an inwardly directed force to the resilient prongs 191, 192.

FIG. 8A illustrates a longitudinal cross-section of a distal portion of the medical grasping device 100. As shown in FIG. 8A, the elongate member 160 is concentrically disposed within the filler tube 170 and the stem 195 of the grasping member 190. The filler tube 170 is concentrically disposed within the catheter 180. A distal end of the filler tube 170 is disposed proximal to a proximal end of the grasping member 190. The first resilient prong 191 extends from a distal end of the catheter 180 in the open state. A grasper closing member or catheter tip 181 is disposed at the distal end of the catheter 180. The grasper closing member 181 can be a metal tube coupled to the distal end of the catheter 180. The catheter 180 may include a reinforcement braid or coil of metal or fabric strands configured to increase tensile strength and kink resistance of the catheter 180. In some embodiments, the reinforcement braid can extend over the grasper closing member 181 to enhance the securement of the grasper closing member 181 to the catheter 180. As depicted in FIG. 8B, when the catheter 180 is displaced distally by the slide actuator 110, the catheter 180 and the grasper closing member 181 extends over the grasping member 190 causing the grasping member 190 to transition from the open state to the closed state. This transition can occur without longitudinal displacement (e.g., proximal displacement) of the grasping member 190 resulting in enhanced accuracy of grasping and retrieval of the target foreign body as shown in FIG. 9.

FIG. 9 illustrates a method of use of the medical grasping device 100. The medical grasping device 100 is introduced into a body cavity through a percutaneously inserted vascular sheath. When passed through the vascular sheath, the medical grasping device 100 is in the closed state wherein the slide button 120 is in a distal position and the grasping member 190 is disposed within the catheter 180 and grasper closing member 181. The distal end of the catheter 180 is directed toward a target foreign body or object 95. The target foreign body can be any one of an inferior vena cava filter, a pacer lead, a vascular stent, a loop, and a suture. Other foreign bodies are within the scope of this disclosure. When the grasping member 190 is positioned adjacent or near the target foreign object or body 95, the slide button 120 is depressed to disengage the locking teeth 113, 132. The slide button 120 is displaced proximally causing the catheter 180 to be displaced proximally. The grasping member 190 remains longitudinally stagnant and transitions from the closed state to the open state as it is exposed from the catheter 180. The slide button 120 is released resulting in the locking member 130 to be biased upward causing the locking teeth 113, 132 to engage to prevent the catheter from longitudinal movement.

The grasping member 190 is manipulated through manipulation of the slide actuator wherein the first and second grasping features 193, 194 are disposed beyond at least a portion of the target foreign object 95. The slide button 120 is depressed and displaced distally wherein the catheter 180 and grasper closing member 181 is displaced over the grasping member 190 while the grasping member 190 remains longitudinally stagnant. The first and second grasping features close together around at least a portion of the target foreign object 95. In another embodiment, the slide button 120 is displaced distally without depression wherein the slide button 120 ratchets along the locking teeth 113, 132 as the slide button 120 is moved distally.

The slide actuator 110 is moved or pulled proximally causing the at least a portion of the target foreign body 95 to be grasped by or engaged by the proximal faces 199a, 199b of the grasping features 193, 194. The grasping member 190 is anchored to the slide actuator 110 via the elongate member 160 and the anchor 150 wherein a pull force of at least 10 lbf can be achieved to retrieve the target foreign body 95.

FIG. 10 depicts an embodiment of a medical grasping or retrieving device 200 that resembles the medical grasping or retrieving device 100 described above in certain respects. Accordingly, like features are designated with like reference numerals, with the leading digit incremented to “2.” For example, the embodiment depicted in FIG. 10 includes a catheter 280 that may, in some respects, resemble the catheter 180 of FIG. 1. Relevant disclosure set forth above regarding similarly identified features thus may not be repeated hereafter. Moreover, specific features of the medical grasping device 100 and related components shown in FIGS. 1-9 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical grasping device 200 and related components depicted in FIG. 10. Any suitable combination of the features, and variations of the same, described with respect to the medical grasping device 100 and related components illustrated in FIGS. 1-9 can be employed with the medical grasping device 200 and related components of FIG. 10, and vice versa. This pattern of disclosure applies equally to further embodiments depicted in subsequent figures and described hereafter, wherein the leading digits may be further incremented.

As illustrated in FIG. 10, the medical grasping or retrieving device 200 includes a slide actuator 210, a catheter 280 operationally coupled to the slide actuator 210, and a grasping or retrieving member 290. In the depicted embodiment of FIG. 10, the catheter 280 includes a bend 282 wherein a distal portion of the catheter 280 and the grasping member 290 are not in axial alignment with a longitudinal axis of the medical grasping device 200. The bend 282 can include an angle β ranging from about 0 degrees to about 90 degrees, from about 20 degrees to about 60 degrees, from about 15 degrees to about 40 degrees, and may be about 30 degrees. The bend 282 may facilitate directing a distal end of the catheter 280 toward a target foreign body or object.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of retrieving a foreign object from a body lumen may include one or more of the following steps: disposing a grasping device to a position within a body lumen adjacent a target foreign object; actuating the slide actuator such that the first and second grasping features overlap with each other to secure the engaged target foreign object; and longitudinally proximally displacing the grasping device to dislodge the secured target foreign object from a position within the body lumen. Other steps are also contemplated.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The terms “proximal” and “distal” refer to opposite ends of a medical device, including the devices disclosed herein. As used herein, the proximal portion of a medical device is the portion nearest a practitioner during use, while the distal portion is a portion at the opposite end. For example, the proximal end of a medical grasping or retrieving device is defined as the end closest to the practitioner during insertion or utilization of the medical grasping or retrieving device. The distal end is the end opposite the proximal end, along the longitudinal direction of the medical grasping or retrieving device.

The terms “foreign body” and “foreign object” refer to any item, matter, or substance, such as a medical device or an embolism, which may be disposed or positioned within a body lumen. In some embodiments, the foreign body or object may be undesirable or unwanted. Specifically, a foreign body or object may be an item that a practitioner desires or targets to remove or retrieve from within a body lumen. For example, the foreign body or object may comprise a medical device (e.g., a vascular filter or a stent) that is disposed within a vessel, and a medical grasping or retrieving device of the present disclosure may be configured to retrieve the medical device from within the vessel. Further, disclosure herein relating to displacement of foreign objects may analogously be applied to any target for displacement or removal, including, for example, bodily structures or materials.

The term “resilient” refers to a component, device, or object that is formed with a particular shape, that can then be elastically deformed into a different shape, but that can return to the original shape when unconstrained. For example, a resilient prong may be formed with a first shape, the resilient prong may then be constrained (i.e., disposed within a lumen of a sheath) to elastically deform it into a second shape, then unconstrained (i.e., displaced out of the lumen of the sheath) such that the resilient prong returns to its first shape. Shape memory alloys, including NITINOL, are examples of resilient materials.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially square” is recited with respect to a feature, it is understood that in further embodiments, the feature can have a precisely square configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical grasping device for use in an interventional procedure in a body lumen of a patient, the medical grasping device comprising:
a slide actuator comprising:
a housing;
a slide button slidingly coupled to the housing;
a locking member coupled to the slide button and releasably lockingly engageable to the housing; and
a slide carriage coupled to the slide button and the locking member and slidingly disposed within the housing;
a catheter coupled to the locking member and distally extendible relative to the housing;
an elongate member concentrically disposed within the catheter, wherein the catheter is slidable over the elongate member; and
a grasping member having an open state and a closed state, wherein the catheter is disposed over the grasping member when the grasping member is in the closed state.

2. The medical grasping device of claim 1, further comprising an anchor coupled to the elongate member and the housing.

3. The medical grasping device of claim 1, further comprising a resilient member coupled to the slide button and the locking member, wherein the resilient member forces the locking member into locking engagement with the housing.

4. The medical grasping device of claim 1, further comprising a filler tube concentrically disposed within the catheter and over the elongate member.

5. The medical grasping device of claim 1, wherein the grasping member comprises:
a stem couplable with the elongate member;
a first resilient prong extending from the stem;
a second resilient prong extending from the stem;
a first grasping feature coupled to the first resilient arm; and
a second grasping feature coupled to the second resilient arm.

6. The medical grasping device of claim 5,
wherein the first grasping feature is oriented radially inward and angled proximally,
wherein the second grasping feature is oriented radially inward and angled proximally, and
wherein the first grasping feature and the second grasping feature are configured to overlap when the grasping member is in the closed state.

7. The medical grasping device of claim 6,
wherein the first grasping feature is angled proximally by negative 5 degrees to 20 degrees, and
wherein the second grasping feature is angled proximally by negative 5 degrees to 20 degrees.

8. The grasping device of claim 1, wherein the catheter comprises:
a tube comprising a reinforcement braid; and
a closing member disposed at a distal end of the tube, wherein the reinforcement braid is coupled to the closing member and wherein the grasping member is disposable within the closing member when in the closed state.

9. The grasping device of claim 8, wherein the closing member comprises a metal material.

10. The grasping device of claim 1, wherein the catheter comprises a bend ranging from 20 degrees to 60 degrees relative to a longitudinal axis of the catheter.

11. A medical retrieval system for use in an interventional procedure in a body lumen of a patient, the medical retrieval system comprising:
a medical retrieval device comprising:
a slide actuator;
a catheter distally extendible relative to the slide actuator;
an elongate member concentrically disposed within the catheter, wherein the catheter is slidable over the elongate member; and
a retrieval member having an open state and a closed state, wherein the retrieval member is at least partially disposed within the catheter when in the closed state; and
an introducer sheath configured for passage of the catheter.

12. The medical retrieval system of claim 11, wherein the retrieval member comprises:
a stem couplable with the elongate member;
a first resilient arm extending from the stem;
a second resilient arm extending from the stem;
a first retrieval feature coupled to the first resilient arm; and
a second retrieval feature coupled to the second resilient arm.

13. The medical retrieval system of claim 12,
wherein the first retrieval feature is oriented radially inward and angled proximally,
wherein the second retrieval feature is oriented radially inward and angled proximally, and
wherein the first grasping feature and the second grasping feature are configured to overlap when the grasping member is in the closed state.

14. The medical retrieval system of claim 13,
wherein the first retrieval feature is angled proximally by negative 5 degrees to 20 degrees, and
wherein the second retrieval feature is angled proximally by negative 5 degrees to 20 degrees.

15. A method of retrieving a foreign object from a body lumen, comprising:
disposing the grasping device of claim 1 to a position within a body lumen adjacent a target foreign object;
actuating the slide actuator such that the first and second grasping features overlap with each other to secure the engaged target foreign object; and
longitudinally proximally displacing the grasping device to dislodge the secured target foreign object from a position within the body lumen.

16. The method of claim 15, wherein actuating the slide actuator comprises:
depressing the slide button;
displacing the slide button distally; and
disposing the catheter over the grasping device.

17. The method of claim 15, further comprising transitioning the grasping member from the open state to the closed state.

18. The method of claim 15, further comprising locking the grasping device in a distally displaced position such that the grasping member is in the closed state.
